(19) United States
(12) Patent Application Publication
(10) Pub. No.: US 2009/0222987 A1
(43) Pub. Date: Sep. 10, 2009

(54) EXAMINATION BED FOR AN MRI SYSTEM

(76) Inventors: Zeng He He, Shenzhen (CN); Yan Hong Chen, Shenzhen (CN); Jian Min Wang, Shenzhen (CN)

Correspondence Address: SCHIFF HARDIN, LLP;PATENT DEPARTMENT, 6600 SEARS TOWER, CHICAGO, IL, 60606-6473, US

(21) Appl. No.: 12/361,742

(22) Filed: Jan. 29, 2009

(30) Foreign Application Priority Data
Jan. 29, 2008 (CN) 20082001566.6

(51) Int. Cl. A61B 6/04

(52) U.S. Cl. 5/601

(57) ABSTRACT

An examination bed for use in a magnetic resonance imaging system, has a bed board, a bottom antenna for an RF coil and a main body antenna for an RF coil. The bottom antenna is integrated in the inside or surface of the bed board, and forms a loop with the main body antenna, to form an RF coil. The thickness of the base plate is thereby reduced by integrating the bottom antenna into the bed board, thus increasing the available space to make the most of the limited space of the magnetic field for examination so that the examination bed can be used for those patients or other tested objects that would otherwise be oversized for the bed.

### BACKGROUND OF THE INVENTION

1. Field of the Invention

The present invention relates to an MRI system, and more particularly to an examination bed used in an MRI system.

2. Description of the Prior Art

Widely used medically, an MRI system usually has a magnet for generating a stable basic magnetic field, a gradient coil for generating a quick on/off gradient field and an RF device, and may further have an examination bed for supporting the patient or tested object. The patient or object to undergo an MRI examination is placed onto the examination bed and the examination bed is moved into the gradient magnetic field generated by the magnet and gradient coil. The RF device transmits (radiates) RF signals into the tested object and receives the generated MR signals. MR images are reconstructed based on these MR signals. In this case, the RF device is an RF coil for transmitting and receiving signals and an associated control system. Depending on the structure and the purpose of the examination, the RF coil may be categorized into head coil, body coil, knee and ankle coil, pliable surface coil, and special coils for the breasts, rectal cavity and uterine cavity, etc. Of these coils, the body coil is one of the most frequently used coils and has a shape and size appropriate for examination such human parts as the trunk and thigh.

As shown in FIG. 1, the system for magnetic resonance imaging has an examination bed 1, a magnet 2 and a body coil 3. The magnet 2 can be a C-shaped permanent magnet as illustrated in FIG. 1. In this case, the examination bed further has a bed board 11. As shown in FIG. 2, the body coil comprises a base plate 31, the upper side of which is a U-shaped main body antenna 32 used to generate RF signals. The base plate 31 has a built-in bottom antenna 33 which forms a loop together with the U-shaped main body antenna 32. There is an RF transmitter coil 4 inside the magnet 2 located under the examination bed 1. Since the base plate 31 has the built-in bottom antenna 33, the existing base plate 31 for the body coil is relatively thick, generally up to 30 millimeters in thickness.

As shown in FIG. 1, to perform the MRI exam, the body coil 3 placed on the examination bed 1. To implement the scanning, the patient or tested object is positioned on the base plate 31 for the body coil and then the U-shaped main body antenna 32 is placed on the base plate 31 so that the U-shaped main body antenna 32 and the bottom antenna 33, which is built into the base plate 31, form a loop. Next, the examination bed 1, together with the body coil 3 and patient or tested object thereon, is moved into the magnetic field generated by the magnet 2. The RF transmitter coil 4 transmits high frequency signals and the body coil 3 receives the signals emitted from the patient or tested object in response.

The examined patient or object is located in the examination space encompassed by the U-shaped main body antenna 32 and the base plate 31. Because this space is limited, some obese patients who are oversized for the bed or other oversized objects cannot be examined. Also, since the interior space formed by the basic field magnet is limited, it is very difficult to increase the examination space by increasing the size of the magnet interior, and it is costly and very uneconomical to redesign or modify the overall shape and size of the magnet.

### SUMMARY OF THE INVENTION

An object of the present invention is to provide an examination bed for use in an MRI system that fully utilizes the available space of the magnetic field for an examination.

This object is achieved in accordance with the invention by an examination bed for an MRI system having a bed board, a bottom antenna for an RF coil and a main body antenna for an RF coil, wherein the bottom antenna is integrated in the inside of the bed board and forms a loop with the main body antenna for an RF coil.

Preferably, the bottom antenna can also be integrated in the upper side or underside of the bed board of the examination bed. The RF coil can be a body coil with a U-shaped main body antenna.

Preferably, the MRI system is a permanent MRI system, which has a vertically oriented magnetic field. In this case, the MRI system has a magnet that can also be a C-shaped permanent magnet. There is an RF transmitter coil inside the magnet under the examination bed.

In the examination bed according to the invention, the conventionally-used base plate used for integrating the bottom antenna has effectively been “removed” by integrating the bottom antenna for an RF coil into the bed board of the examination bed, which ensures that the mechanical strength of the bed board of the examination bed is still maintained. Moreover, when the main body antenna for the body coil is not used, it does not form a loop with the bottom antenna that is integrated into the bed board of the examination bed and does not influence the operation of other coils such as the head coil.

By integrating the bottom antenna for coils into the bed board of the examination bed, the thickness of the base plate for the existing body coil can be reduced, thus increasing the available space inside the coil to fully utilize the limited space of the magnetic field. In this way, the examination bed can be used for those patients or other tested objects that would otherwise exceed its limit.

### DESCRIPTION OF THE DRAWINGS

FIG. 1 is a schematic diagram showing the operating state of the body coil of the prior art when used in a permanent MRI system.

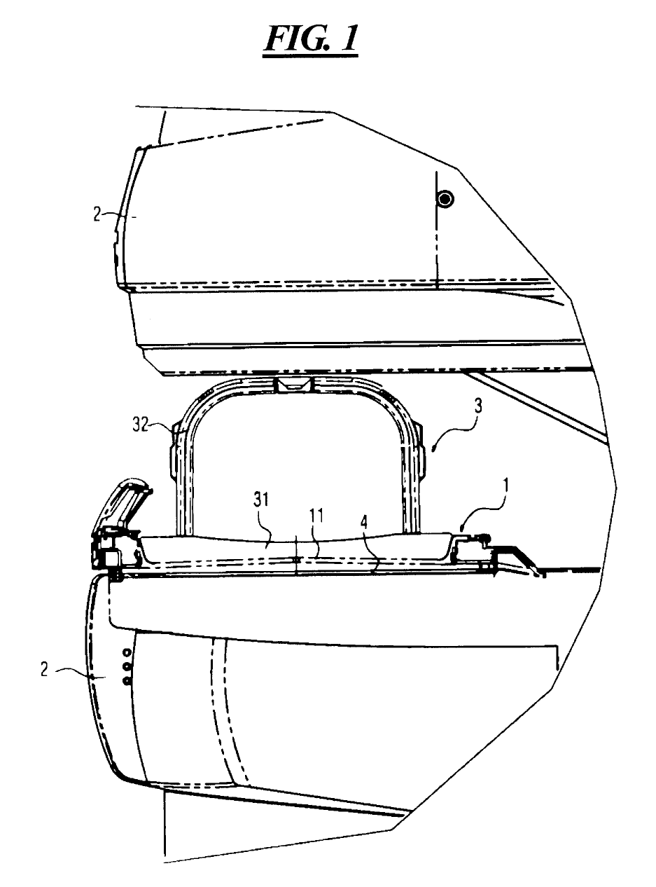

FIG. 2 shows the structure of the body coil in the prior art.

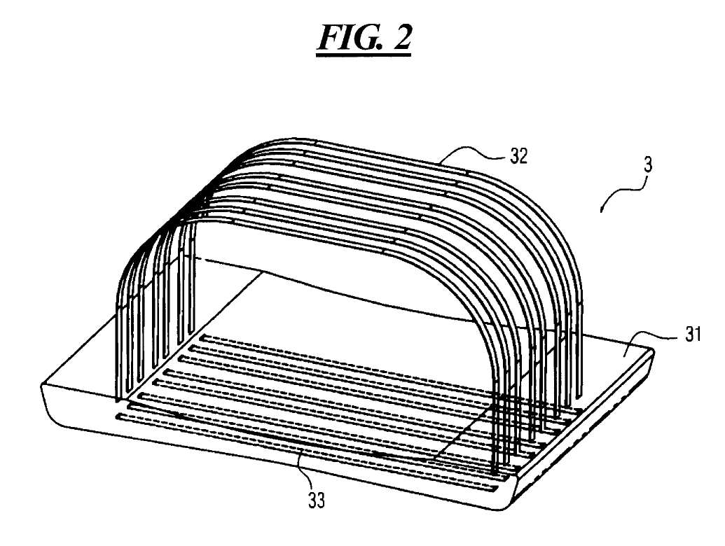

FIG. 3 is a schematic diagram showing the work status of the examination bed for an MRI system in a preferred embodiment according to the present invention.

FIGS. 4 and 5 are schematic diagrams showing the examination bed for an MRI system in a preferred embodiment according to the present invention.

### DESCRIPTION OF THE PREFERRED EMBODIMENTS

In order to make the technical features, purposes and results of the present utility model more evident and understandable, the embodiments of the present utility model are described in detail with reference to figures. In the figures, the same components are denoted by the same reference number.

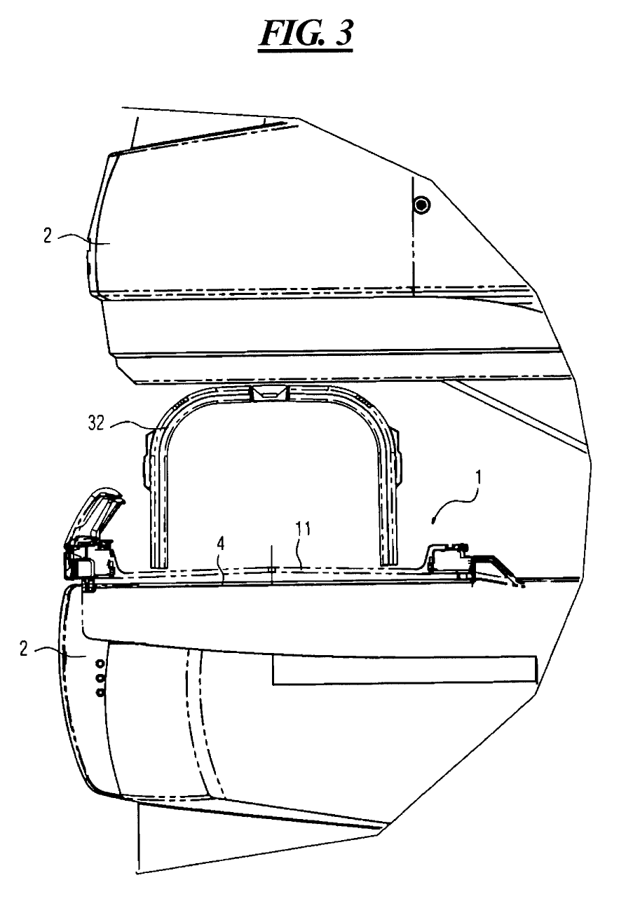

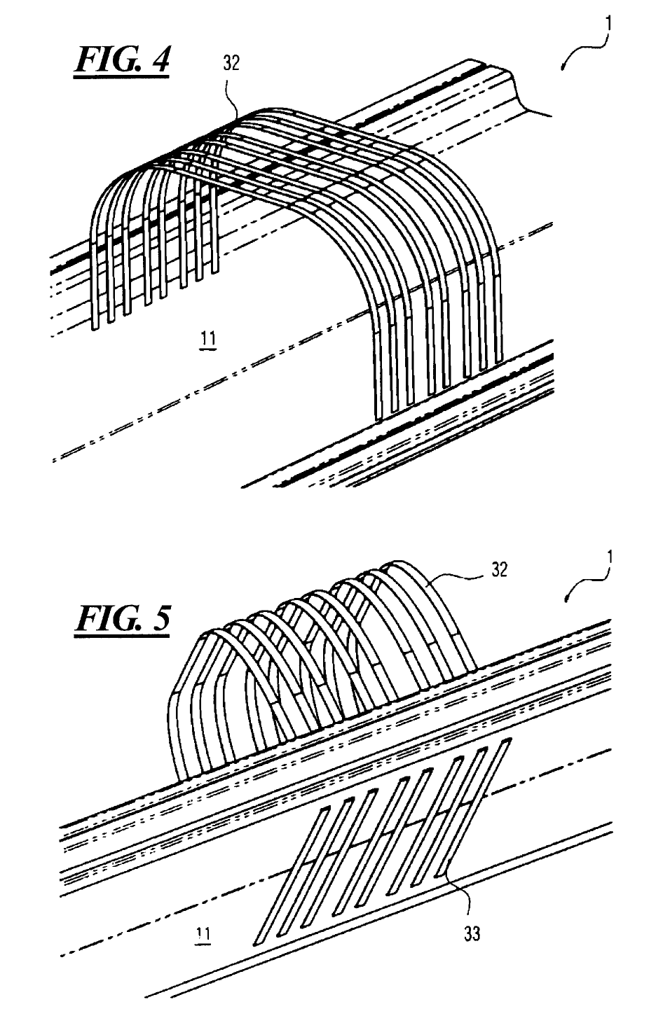

In the embodiment of the invention shown in FIG. 3, the MRI system has an examination bed 1 and a magnet 2. In a preferred embodiment, the magnet 2 is a C-shaped permanent magnet; of course, the magnet can also be a superconducting magnet with a vertically oriented magnetic field. Referring to both FIG. 4 and FIG. 5, the examination bed has a bed board 11 of the examination bed, a bottom antenna 33 for an RF coil and a main body antenna 32 for an RF coil. The bottom antenna 33 is directly integrated into the inside of the bed board 11 and, together with the main body antenna 32, forms an RF coil. An RF transmitter coil 4 is provided on the inside of the magnet under the examination bed 1.

In a preferred embodiment, the RF coil is a body coil and the main body antenna of the RF coil is substantially U-shaped. Those of ordinary skill in the art will understand that the RF coil can also be for special purpose coils other than a body coil, for example, a head coil or knee and ankle coils. Moreover, the main body antenna for an RF coil can be in other shapes, for example, C-shaped.

In a preferred embodiment, the bottom antenna 33 for an RF coil is integrated into the inside of the bed board 11 of the examination bed 1 for an MRI system. Those of ordinary skill in the art will understand that the bottom antenna 33 can also be integrated into the upper side or underside of the bed board 11.

When the MRI exam is performed, the patient lies directly onto the bed board 11 of the examination bed with integrated bottom antenna 33 for the body coil, and then the U-shaped main body antenna 32 for the body coil is placed onto the bed board 11 of the examination bed and is aligned and connected with the bottom antenna 33 to form a loop, constituting a body coil. Next, the examination bed 1 is moved into the magnetic field, the RF transmitter coil 4 transmits HF signals, and the body coil receives the signals fed back from the patient. When the body coil is not used, the main body antenna 32 will not be placed, and the bottom antenna 33, which is integrated into the bed board 11 of the examination bed 1, cannot form a loop and cannot influence the operation of other coils, for example, the head coil.

As shown in FIG. 4 and FIG. 5, unlike the prior art, a major feature of the examination bed for MRI system according to the present utility model is that it eliminates the base plate that is integrated with the bottom antenna.

During actual operation, the main body antenna 32 and bed board 11 form an enveloping space and the tested patient or object is located in this enveloping space. As described above, conventionally the bottom antenna 33 is integrated into the base plate 2 which thus must necessarily be made thicker. Therefore, unlike the present utility model, the base plate 2 will occupy a portion of the examination space, and sometimes may be up to about 30 millimeters in thickness, making the examination bed unsuitable for obese patients or other tested objects that exceed the size limit. In accordance with the invention, since the bottom antenna 33 for an RF coil is directly integrated into the bed board 11, at least some additional space can be gained. Although the gained space may not be large, it makes modification of the existing equipment easier and the costs are lower than modification of the magnets. The integrated bed board of the examination bed is around 10 millimeters thick and its mechanical strength remains the same.

By integrating the bottom antenna 33 for an RF coil into the bed board 11 of the examination bed 1, the space inside the coil which is occupied by the thickness of the base plate 2 of the existing body coil is spared, thus increasing the available space and allowing some obese patients or other tested objects that would otherwise exceed the size limit to be examined.

Although modifications and changes may be suggested by those skilled in the art, it is the intention of the inventors to embody within the patent warranted hereon all changes and modifications as reasonably and properly come within the scope of their contribution to the art.

We claim as our invention:

1. An examination bed used in the magnetic resonance imaging (MRI) system, comprising a bed board, a bottom antenna for an RF coil and a main body antenna for an RF coil, said bottom antenna for an RF coil is integrated in the inside of the bed board and forming a loop with said main body antenna to form said RF coil.

2. The examination bed as claimed in claim 1, wherein said bottom antenna is integrated in the upper side of the bed board.

3. The examination bed as claimed in claim 1, wherein said bottom antenna is integrated in the underside of the bed board.

4. The examination bed as claimed in claim 1, wherein said RF coil is a body coil.

5. The examination bed as claimed in claim 4, wherein said main body antenna for said body coil is U-shaped.

6. The examination bed as claimed in claim 1, wherein said MRI system is a permanent MRI system.
